(19) United States
(12) Patent Application Publication
(10) Pub. No.: US 2023/0092734 A1
(43) Pub. Date: Mar. 23, 2023

(54) TRANSPARENT TATTOO STENCIL AND METHOD THEREOF

(71) Applicant: Brandon Lee Henderson, Nashville, TN (US)

(72) Inventor: Brandon Lee Henderson, Nashville, TN (US)

(21) Appl. No.: 17/948,322

(22) Filed: Sep. 20, 2022

Related U.S. Application Data
(60) Provisional application No. 63/245,961, filed on Sep. 20, 2021.

(51) Int. Cl. A61M 37/00; B41K 3/20

(52) U.S. Cl. CPC A61M 37/0076; B41K 3/20

(57) ABSTRACT

A transparent tattoo stencil transfer and associated method are provided herein. The transparent tattoo stencil transfer allows a tattoo artist to transfer a selected design onto a transparent plastic sheet for accurate and precise placement of the design from the transparent plastic sheet onto a client's skin. The transparent tattoo stencil transfer may comprise at least a backing sheet, a polyester carbon sheet positioned on top of the backing sheet, a plastic sheet positioned on top of the polyester carbon sheet, and a semi-transparent tissue sheet positioned on top of the plastic sheet. A tattoo design may be placed between the backing sheet and the polyester carbon sheet. A heat transfer process is performed using a thermofax machine to transfer a carbon image of the tattoo design onto the plastic sheet using the polyester carbon sheet.

### CROSS-REFERENCES TO RELATED APPLICATIONS

This application claims benefit of the following patent application which is hereby incorporated by reference: U.S. Provisional Application No. 63/245,961 filed Sep. 20, 2021, entitled “Transparent Tattoo Stencil and Method Thereof.”

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND

### 1. Field of the Invention

The present invention relates generally to tattoo stencils.

More particularly, this invention pertains to the transfer of tattoo stencils onto an area of skin for tattooing.

### 2. Description of the Prior Art

Referring to FIG. 1, a typical four-layer transfer paper 100 is shown. The four-layer transfer paper 100 comprises a yellow backing sheet 102, a polyester carbon sheet 104, a semi-transparent tissue sheet 106, and a white master sheet 108. Generally, once a tattoo design is selected, it is placed between the yellow backing sheet 102 and the polyester carbon sheet 104. The four-layer transfer paper 100 and the selected design are then loaded into a “thermo carrier” and run through a thermofax machine. The thermofax machine uses heat to transfer the selected design onto the white master sheet 108 using the polyester carbon sheet 104. The selected tattoo design may be custom drawn or may be selected from a catalog.

An issue arises when it comes time to transfer the design from the white master sheet 108 to a client's skin for tattooing. Properly placing the design is incredibly difficult since a tattoo artist cannot see through the white master sheet 108. The tattoo artist must hope that the design is in the perfect location while trying to see through or peer around the edges of the white master sheet 108. If the design is not placed correctly, the tattoo artist has to spend time cleaning off and replacing the design, which may again be misaligned. This problem is compounded when it comes to cover up tattoos. In the case of cover up tattoos, the artist creates a custom and properly sized design that works to cover up a previously tattooed and unwanted design. Precise placement is of the utmost importance when it comes to cover up tattoos.

### BRIEF SUMMARY

In view of at least some of the above-referenced problems, an exemplary object of the present disclosure is to provide a transparent transfer stencil. The transparent tattoo stencil replaces the white master sheet as the medium for transferring the design onto the client's skin for tattooing. The transparent transfer stencil provides added precision when placing the design since the visual obstruction caused by the white master sheet is removed.

The transparent transfer stencil would primarily be used in the rapidly growing tattoo industry but would also be used by salons that offer cosmetic tattooing.

The transparent transfer stencil benefits a tattoo artist by giving them the means to precisely place designs customized to perfectly fit the exact area the client is looking to have tattooed. The transparent transfer stencil also allows artists to design more complex cover ups and further eliminates the hassle of replacing the design multiple times.

In a particular embodiment, an exemplary transparent tattoo stencil transfer as disclosed herein may include a backing sheet, a carbon sheet configured to be positioned on top of the backing sheet, and a plastic sheet configured to be positioned on top of the carbon sheet. In accordance with this embodiment, a tattoo design is configured to be received between the backing sheet and the carbon sheet. Further in accordance with this embodiment, the carbon sheet is configured to transfer a carbon image of the tattoo design onto the plastic sheet during a heat transfer process.

In an exemplary aspect according to the above-referenced embodiment, the transparent tattoo stencil transfer may further comprise at least one semi-transparent tissue sheet positioned above the carbon sheet.

In another exemplary aspect according to the above-referenced embodiment, two or more of the backing sheet, the carbon sheet, the plastic sheet, or the at least one semi-transparent tissue sheet may be bound together along a common edge, respectively, to define a binding.

In another exemplary aspect according to the above-referenced embodiment, the backing sheet and the carbon sheet may be bound together along a common edge, respectively, to define a binding.

In another exemplary aspect according to the above-referenced embodiment, the plastic sheet is positioned between the backing sheet and the carbon sheet prior to the heat transfer process such that it is protected during shipping of the transparent tattoo stencil transfer. In accordance with this aspect, the plastic sheet may be repositioned on top of the carbon sheet during or prior to the heat transfer process.

In another exemplary aspect according to the above-referenced embodiment, the binding may comprise glue.

In another exemplary aspect according to the above-referenced embodiment, the plastic sheet has a transmittance value above 50%.

In another exemplary aspect according to the above-referenced embodiment, a shipping configuration may be defined by the plastic sheet being removably positioned between the backing sheet and the carbon sheet. In accordance with this aspect, a heat transfer configuration may be defined by the plastic sheet being positioned on top of the carbon sheet.

In another exemplary aspect according to the above-referenced embodiment, at least one semi-transparent tissue sheet may be positioned above the carbon sheet in the shipping configuration for protecting the carbon sheet.

In another exemplary aspect according to the above-referenced embodiment, at least one semi-transparent tissue sheet may be removed from between the carbon sheet and the plastic sheet in the heat transfer configuration.

In another exemplary aspect according to the above-referenced embodiment, at least one of a white sheet or a semi-transparent tissue sheet may be positioned above the carbon sheet.

In another embodiment, a method of utilizing a transparent tattoo stencil transfer as disclosed herein may include the steps of (a) providing the transparent tattoo stencil transfer comprising at least a backing sheet, a carbon sheet positioned above the backing sheet, and a plastic sheet positionable above the carbon sheet; (b) positioning a tattoo design between the backing sheet and the carbon sheet; (c) performing a heat transfer process on the transparent tattoo stencil transfer to transfer a carbon image of the tattoo design from the carbon sheet onto the plastic sheet; and (d) positioning the plastic sheet with the carbon image of the tattoo design onto a client's skin for transferring the carbon image of the tattoo design for tattooing.

In an exemplary aspect according to the above-referenced embodiment, step (a) of the method may further comprise repositioning the plastic sheet from a shipping configuration between the backing sheet and the carbon sheet to a heat transfer configuration on top of the carbon sheet.

In another exemplary aspect according to the above-referenced embodiment, the transparent tattoo stencil transfer may further comprise at least one semi-transparent tissue sheet positioned above the carbon sheet for protecting the carbon sheet in a shipping configuration.

In another exemplary aspect according to the above-referenced embodiment, prior to step (c), the method may further comprise removing the at least one semi-transparent tissue sheet.

In another exemplary aspect according to the above-referenced embodiment, two or more of the backing sheet, the carbon sheet, or the at least one semi-transparent tissue sheet may be bound together along a common edge, respectively, to define a binding. In accordance with this aspect, the binding may comprise glue.

In another exemplary aspect according to the above-referenced embodiment, step (c) of the method may further comprise feeding the transparent tattoo stencil transfer with the tattoo design through a thermofax machine. In accordance with this aspect, the method may further comprise positioning the transparent tattoo stencil transfer with the tattoo design into a thermo carrier prior to feeding the transparent tattoo stencil transfer with the tattoo design through the thermofax machine.

In another exemplary aspect according to the above-referenced embodiment, step (d) of the method may further comprise removing the plastic sheet with the carbon image from the transparent tattoo stencil transfer.

In another exemplary aspect according to the above-referenced embodiment, step (d) of the method may further comprise transferring the carbon image from the plastic sheet into the client's skin.

### BRIEF DESCRIPTION OF THE SEVERAL VIEWS OF THE DRAWINGS

FIG. 1 is a perspective diagram of a typical four-layer transfer paper.

FIG. 2 is a perspective diagram of an embodiment of a transparent tattoo stencil in accordance with the present disclosure.

FIG. 3 is a perspective diagram of another embodiment of a transparent tattoo stencil in accordance with the present disclosure.

FIG. 4 is a perspective diagram of another embodiment of a transparent tattoo stencil in accordance with the present disclosure.

FIG. 5 is a perspective diagram of another embodiment of a transparent tattoo stencil in accordance with the present disclosure.

FIG. 6 is a perspective diagram of another embodiment of a transparent tattoo stencil in accordance with the present disclosure.

FIGS. 7-12 are illustrative steps of an embodiment of a method of utilizing the transparent tattoo stencil in accordance with the present disclosure.

FIG. 13 is a flow diagram of the method as illustrated in FIGS. 7-12 in accordance with the present disclosure.

FIG. 14 is a flow diagram of another embodiment of a method of utilizing the transparent tattoo stencil in accordance with the present disclosure.

### DETAILED DESCRIPTION

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure.

The words “connected”, “attached”, “joined”, “mounted”, “fastened”, and the like should be interpreted to mean any manner of joining two objects including, but not limited to, the use of any fasteners such as screws, nuts and bolts, bolts, pin and clevis, and the like allowing for a stationary, translatable, or pivotable relationship; welding of any kind such as traditional MIG welding, TIG welding, friction welding, brazing, soldering, ultrasonic welding, torch welding, inductive welding, and the like; using any resin, glue, epoxy, and the like; being integrally formed as a single part together; any mechanical fit such as a friction fit, interference fit, slidable fit, rotatable fit, pivotable fit, and the like; any combination thereof; and the like.

Unless specifically stated otherwise, any part of the apparatus of the present disclosure may be made of any appropriate or suitable material including, but not limited to, metal, alloy, polymer, polymer mixture, wood, composite, or any combination thereof.

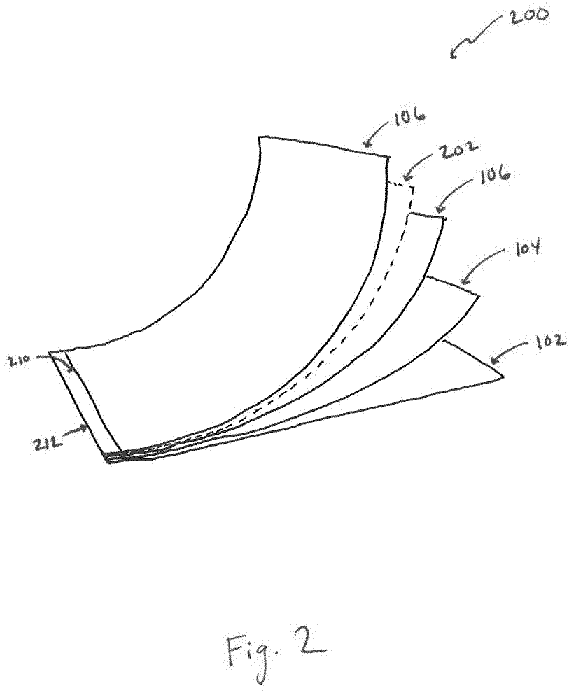

Referring to FIG. 2-6, a transparent tattoo stencil transfer 200 is provided. The transparent tattoo stencil transfer 200 comprises at least the yellow backing sheet 102, the polyester carbon sheet 104, and a plastic sheet 202. The yellow backing sheet 102 may be configured to receive a tattoo design 204. The plastic sheet 202 may be placed directly above the polyester carbon sheet 104 with the yellow backing sheet 102 positioned directly below the polyester carbon sheet 104. The yellow backing sheet 102 may also be referred to herein as a backing sheet 102. The polyester carbon sheet 104 may also be referred to herein as a carbon sheet. The plastic sheet 202 may be clear, however in other embodiments, the plastic sheet 202 may be semi-clear with white or other color tint.

In certain optional embodiments, the transparent tattoo stencil transfer 200 may further comprise at least one semi-transparent tissue sheet 106 positioned above the polyester carbon sheet 104 or the plastic sheet 202. In other optional embodiments, another semi-transparent tissue sheet 106 may be placed between the plastic sheet 202 and the polyester carbon sheet 104 for protecting the plastic sheet 202 during heat transfer and/or preventing accidental carbon transfer prior to heat transfer, for example during shipping, which is further discussed below. In still further optional embodiments, the transparent tattoo stencil transfer 200 may further comprise a white master sheet 108 positioned above the plastic sheet 202 such that the transparent tissue sheet 106 is positioned between the plastic sheet 202 and the white master sheet 108. The white master sheet 108 may be configured to further protect the plastic sheet 202 during heat transfer. In other optional embodiments, the white master sheet 108 may be used in place of yellow backing sheet 102 below polyester carbon sheet 104 and the plastic sheet 202. The benefits associated with adding additional layers above the plastic sheet 202 may include protecting the plastic sheet 202 from overheating while passing through the thermofax machine 208, preventing the plastic sheet 202 from sticking to the thermal carrier 206, and/or protecting any of the various sheets during shipping or the heat transfer process 230, as further discussed below. Various other combinations and/or layer orderings will be apparent based on the following discussions.

As illustrated in FIG. 2, an embodiment of the transparent tattoo stencil transfer 200 may include the yellow backing sheet 102, the polyester carbon sheet 104 positioned above the yellow backing sheet 102, and a plastic sheet 202 sandwiched between the at least one semi-transparent tissue sheet 106 above the polyester carbon sheet 104. The uppermost semi-transparent tissue sheet 106 of FIG. 2 may alternatively be replaced with a white master sheet 108.

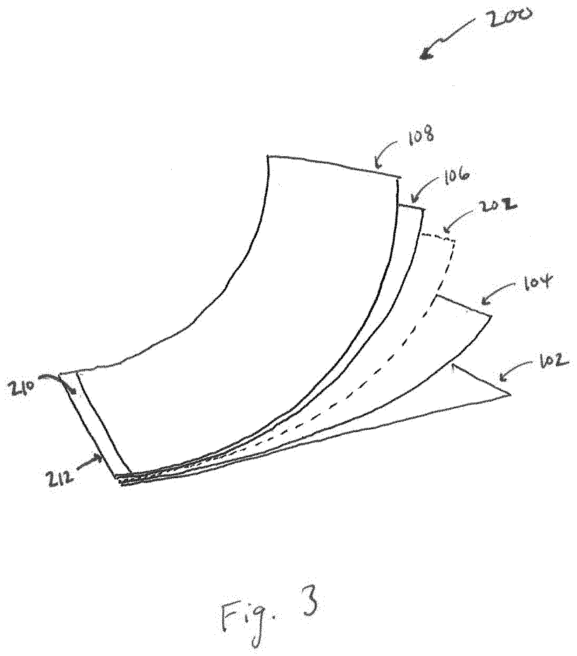

As illustrated in FIG. 3, another embodiment of the transparent tattoo stencil transfer 200 may include the yellow backing sheet 102, the polyester carbon sheet 104 positioned above the yellow backing sheet 102, a plastic sheet 202 positioned above the polyester carbon sheet 104, a semi-transparent tissue sheet 106 positioned above the plastic sheet 202, and a white sheet 108 positioned above the semi-transparent tissue sheet 106. One or more of the semi-transparent tissue sheet 106 or the white sheet 108 may be utilized during the heat transfer process 230 for protecting the plastic sheet 202 or may be removed prior to the heat transfer process 230.

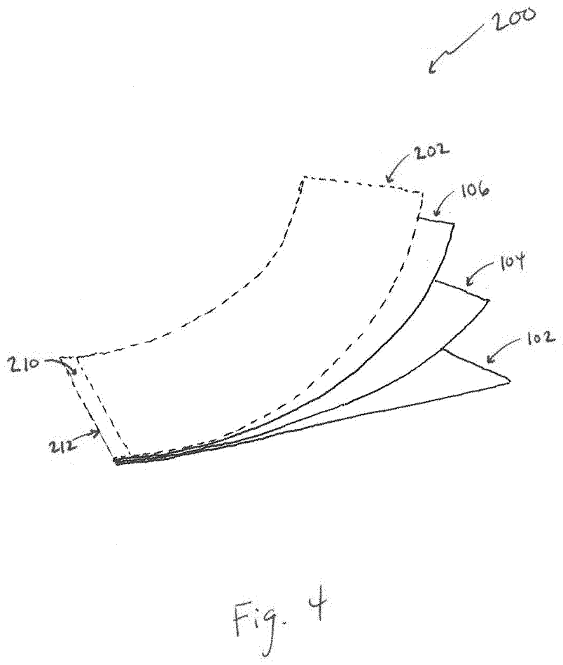

As illustrated in FIG. 4, another embodiment of the transparent tattoo stencil transfer 200 may include the yellow backing sheet 102, the polyester carbon sheet 104 positioned above the yellow backing sheet 102, a semi-transparent tissue sheet 106 positioned above the polyester carbon sheet 104, and a plastic sheet 202 positioned above the semi-transparent tissue sheet 106. The semi-transparent tissue sheet 106 may be utilized to prevent accidental carbon transfer from the polyester carbon sheet 104 prior to the heat transfer process 230, for example during shipping and/or storage of the transparent tattoo stencil transfer 200.

As illustrated in FIG. 5, another embodiment of the transparent tattoo stencil transfer 200 may include the yellow backing sheet 102, the polyester carbon sheet 104 positioned above the yellow backing sheet 102, and a semi-transparent tissue sheet 106 positioned above the polyester carbon sheet 104. In accordance with this embodiment, the plastic sheet 202 may be removably positioned between the yellow backing sheet 102 and polyester carbon sheet 104. This embodiment may define a shipping configuration 240 of the transparent tattoo stencil transfer 200 wherein the plastic sheet 202 is safe from accidental carbon transfer from the polyester carbon sheet 104 prior to the heat transfer process 230, for example during shipping and/or storage of the transparent tattoo stencil transfer 200. The semi-transparent tissue sheet 106 may further protect the polyester carbon sheet 104 in the shipping configuration 240.

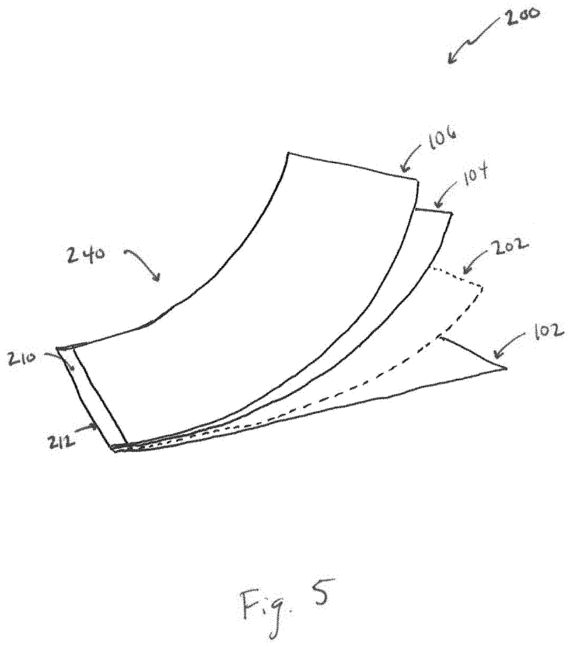

As illustrated in FIG. 6, a heat transfer configuration 242 of the transparent tattoo stencil transfer 200 may be defined by the plastic sheet 202 being positioned (or repositioned) on top of the polyester carbon sheet 104. One or more of the semi-transparent tissue sheet 106 or the white sheet 108 may be removed in the heat transfer configuration 242 in preparation for the heat transfer process 230.

In certain optional embodiments, two or more of the yellow backing sheet 102, the polyester carbon sheet 104, the plastic sheet 202, the at least one semi-transparent tissue sheet 106, or the white sheet 108 may be bound together along a common edge 212, respectively, to define a binding 210. In certain optional embodiments, the binding 210 may comprise glue. In other optional embodiments, the binding 210 may be formed using ultrasonic welding, heat welding, or the like. As illustrated in FIGS. 5-6, the plastic sheet 202 may be repositionable between the various layers and thus may not be bound to the other layers via the binding 210.

The plastic sheet 202 may comprise an archival safe and acid free sheet of plastic. For example, Polypropylene or plastic page protectors are acid-free and archival safe. Another example is cellulose diacetate film which is made from wood pulp and can be recycled, composted or incinerated and is biodegradable and used generally as a film for overlays, color separations and layouts. Other clear or transparent substrates now known or yet to be developed may also be used as the plastic sheet 202.

The plastic sheet 202 may have a transmittance value generally above 50%. In one optional embodiment, the transmittance value may be above 60%. In another optional embodiment, the transmittance value may be above 65%. In another optional embodiment, the transmittance value may be above 70%. In another optional embodiment, the transmittance value may be above 75%. In another optional embodiment, the transmittance value may be above 80%. In another optional embodiment, the transmittance value may be above 85%. In another optional embodiment, the transmittance value may be above 90%. In another optional embodiment, the transmittance value may be above 95%. In another optional embodiment, the transmittance value may be 100%.

The plastic sheet 202 may have a haze value below 50%. In one optional embodiment, the haze value may be below 45%. In another optional embodiment, the haze value may be below 40%. In another optional embodiment, the haze value may be below 35%. In another optional embodiment, the haze value may be below 30%. In another optional embodiment, the haze value may be below 25%. In another optional embodiment, the haze value may be below 20%. In another optional embodiment, the haze value may be below 15%. In another optional embodiment, the haze value may be below 10%. In another optional embodiment, the haze value may be below 5%. In another optional embodiment, the haze value may be 0%.

The transparency of the plastic sheet 202 depends at least in part on the transmittance value and the haze value. As such, for the plastic sheet 202 to have a good transparency, the transmittance value should be kept high while the haze value kept low. A good transparency of the plastic sheet 202 will better enable a tattoo artist or other user of the transparent tattoo stencil transfer 200 to precisely place the plastic sheet 202 with the carbon image 205 of the tattoo design 204 on a client's skin to transfer the design for tattooing.

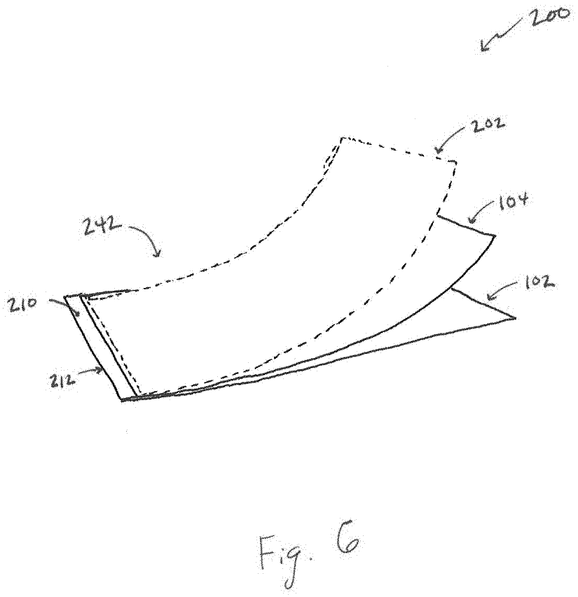

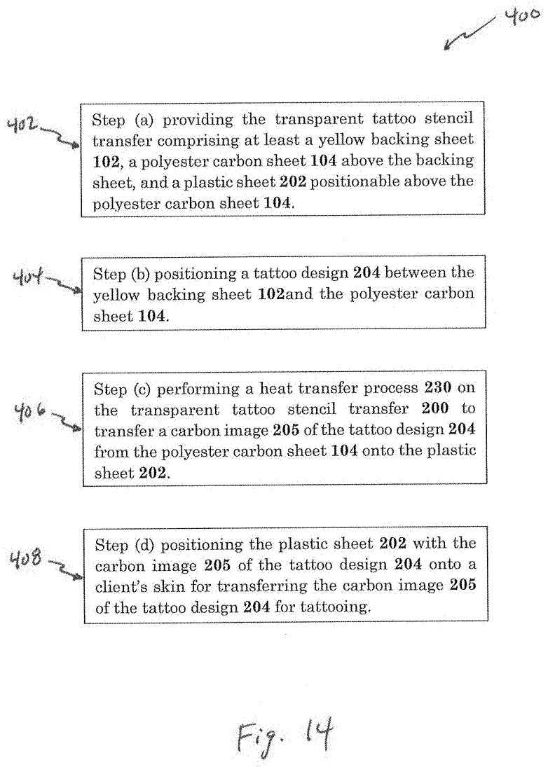

Referring to FIG. 7-12, an embodiment of the various steps of a method 300 of utilizing the transparent tattoo stencil transfer 200 are illustrated. Referring to FIG. 13, a flowchart of the method 300 is provided. As illustrated in FIG. 7, the method 300 may include step (a) selecting 302 a tattoo design 204. The tattoo design 204 may also be referred to herein as a desired design 204. The tattoo design 204 is selected by one or more of the artist or the client.

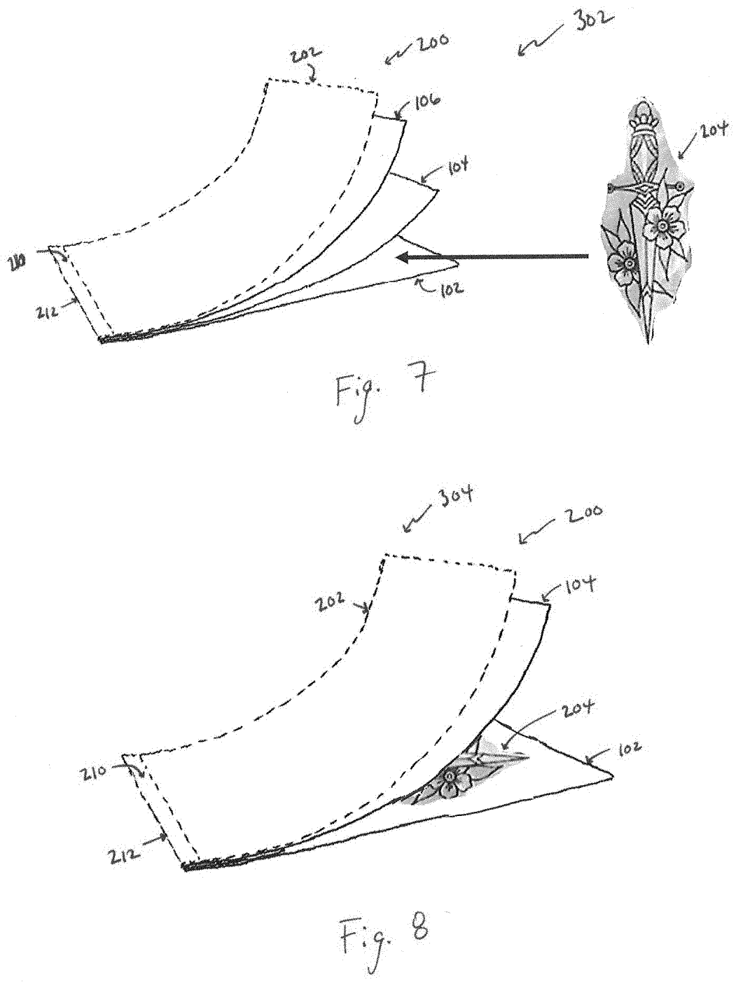

As illustrated in FIG. 8, the method 300 may further include step (b) placing 304 the tattoo design 204 inside the transparent tattoo stencil transfer 200 between the yellow backing sheet 102 and the polyester carbon sheet 104. Step (b) may further comprise facing the tattoo design 204 towards the polyester carbon sheet 104.

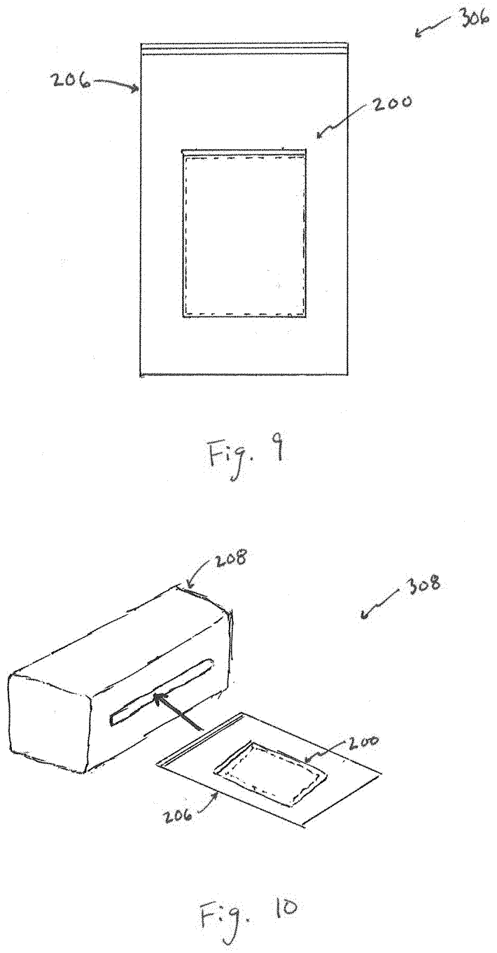

As illustrated in FIG. 9, the method 300 may further include step (c) placing 306 the transparent tattoo stencil transfer 200 with the tattoo design 204 layered therein into a thermo carrier 206.

As illustrated in FIG. 10, the method 300 may further include step (d) feeding 308 the thermo carrier 206 containing the transparent tattoo stencil transfer 200 with the tattoo design 204 layered therein through a thermofax machine 208. Step (d) may further comprise transferring a carbon image 205 of the tattoo design 204 onto the plastic sheet 202 using the polyester carbon sheet 104 and heat from the thermofax machine 208 to accomplish a heat transfer thereof. Step (d) may define, at least partially, the heat transfer process 230 whereby the carbon image 205 of the tattoo design 204 is transferred from the polyester carbon sheet 104 onto the plastic sheet 202.

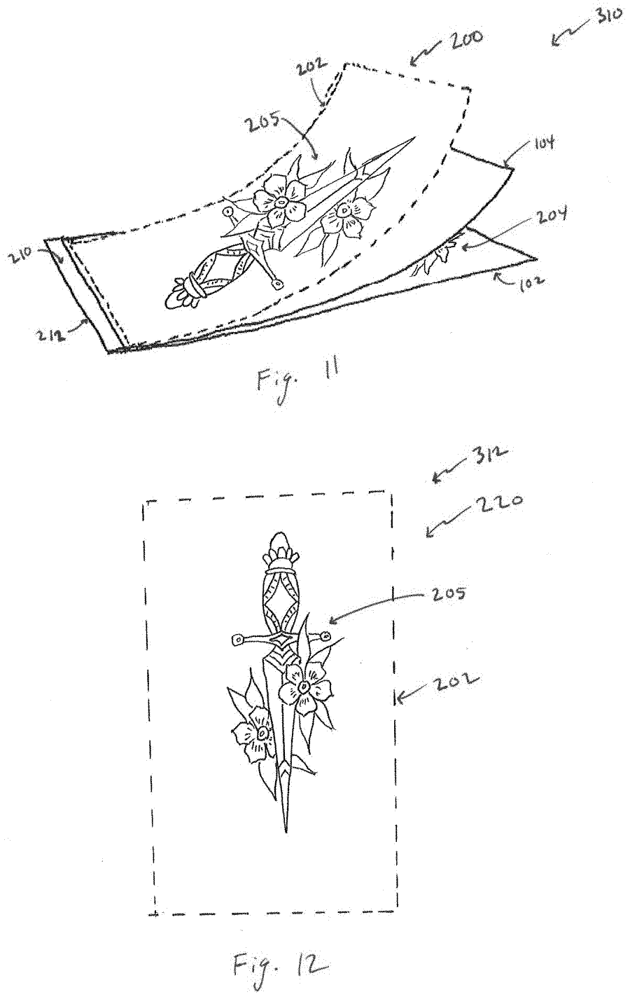

As illustrated in FIG. 11, the method 300 may further include step (e) removing 310 the transparent tattoo stencil transfer 200 with the tattoo design 204 layered therein from the thermo carrier 206.

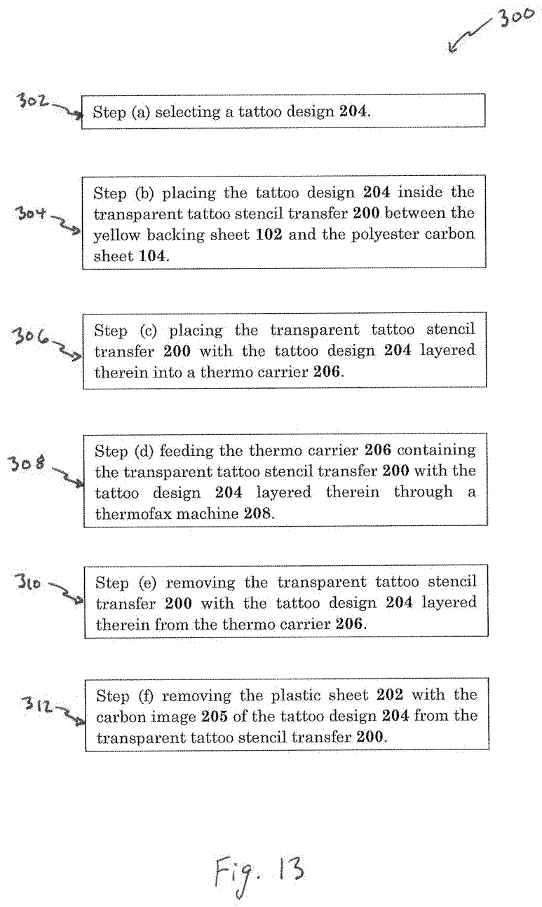

As illustrated in FIG. 12, the method 300 may further include step (f) removing 312 the plastic sheet 202 with the carbon image 205 of the tattoo design 204 from the transparent tattoo stencil transfer 200. The plastic sheet 202 with the carbon image 205 of the tattoo design 204 disposed thereon may also be referred to herein as a transparent tattoo stencil 220. Following step (f), the transparent tattoo stencil 220 is ready to be accurately and precisely placed on a client's skin to transfer the design for tattooing.

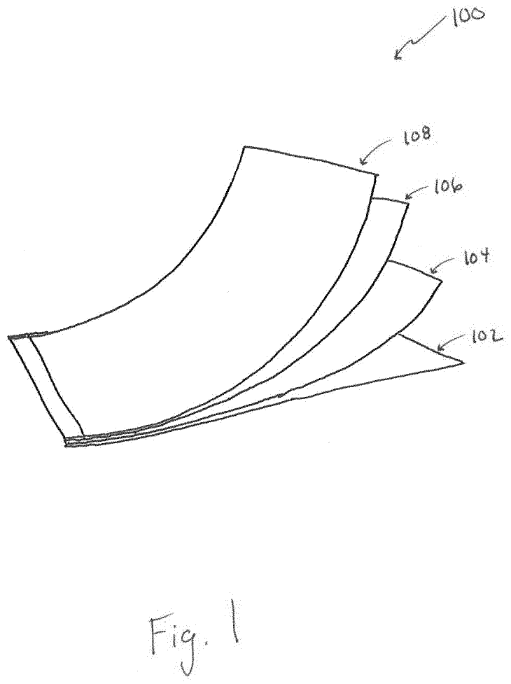

The transparent tattoo stencil 220 eliminates the obstruction and hassle (e.g., not being able to see the desired location) created by the white master sheet 108 (prior art, shown in FIG. 1) which is currently used for transferring designs onto a client's skin for tattooing.

Referring to FIG. 14, another embodiment of a method 400 of utilizing the transparent tattoo stencil transfer 200 is provided. The method 400 may comprise step (a) providing 402 the transparent tattoo stencil transfer comprising at least a yellow backing sheet 102, a polyester carbon sheet 104 above the backing sheet, and a plastic sheet 202 positionable above the polyester carbon sheet 104. The method 400 may further comprise step (b) positioning 404 a tattoo design 204 between the yellow backing sheet 102 and the polyester carbon sheet 104. The method 400 may further comprise step (c) performing 406 a heat transfer process 230 on the transparent tattoo stencil transfer 200 to transfer a carbon image 205 of the tattoo design 204 from the polyester carbon sheet 104 onto the plastic sheet 202. The method 400 may further comprise step (d) positioning 408 the plastic sheet 202 with the carbon image 205 of the tattoo design 204 onto a client's skin (not shown) for transferring the carbon image 205 of the tattoo design 204 for tattooing.

Step (a) of the method 400 may further comprise repositioning the plastic sheet 202 from a shipping configuration 240 between the yellow backing sheet 102 and the polyester carbon sheet 104 to a heat transfer configuration 242 on top of the polyester carbon sheet 104.

The method 400 may further comprise, prior to step (c), removing the at least one semi-transparent tissue sheet 106.

Step (c) of the method 400 may further comprise feeding the transparent tattoo stencil transfer 200 with the tattoo design 204 through a thermofax machine 208. In accordance with this embodiment, the method 400 may further comprise positioning the transparent tattoo stencil transfer 200 with the tattoo design 204 into a thermo carrier 206 prior to feeding the transparent tattoo stencil transfer 200 with the tattoo design 204 through the thermofax machine 208.

Step (d) of the method 400 may further comprise removing the plastic sheet 202 with the carbon image 205 from the transparent tattoo stencil transfer 200.

Step (d) of the method 400 may further comprise transferring the carbon image 205 from the plastic sheet 202 into the client's skin.

Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

Although embodiments of the present invention have been described in detail, it will be understood by those skilled in the art that various modifications can be made therein without departing from the spirit and scope of the invention as set forth in the appended claims.

This written description uses examples to disclose the invention and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It will be understood that the particular embodiments described herein are shown by way of illustration and not as limitations of the invention. The principal features of this invention may be employed in various embodiments without departing from the scope of the invention. Those of ordinary skill in the art will recognize numerous equivalents to the specific procedures described herein. Such equivalents are considered to be within the scope of this invention and are covered by the claims.

All of the compositions and/or methods disclosed and claimed herein may be made and/or executed without undue experimentation in light of the present disclosure. While the compositions and methods of this invention have been described in terms of the embodiments included herein, it will be apparent to those of ordinary skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

The previous detailed description has been provided for the purposes of illustration and description. Thus, although there have been described particular embodiments of a new and useful invention, it is not intended that such references be construed as limitations upon the scope of this disclosure except as set forth in the following claims.

What is claimed is:

1. A transparent tattoo stencil transfer comprising:
a backing sheet;
a carbon sheet configured to be positioned on top of the backing sheet; and
a plastic sheet configured to be positioned on top of the carbon sheet;
wherein a tattoo design is configured to be received between the backing sheet and the carbon sheet, further wherein the carbon sheet is configured to transfer a carbon image of the tattoo design onto the plastic sheet during a heat transfer process.

2. The transparent tattoo stencil transfer of claim 1, further comprising:
at least one semi-transparent tissue sheet positioned above the carbon sheet.

3. The transparent tattoo stencil transfer of claim 2, wherein:
two or more of the backing sheet, the carbon sheet, the plastic sheet, or the at least one semi-transparent tissue sheet are bound together along a common edge, respectively, to define a binding.

4. The transparent tattoo stencil transfer of claim 1, wherein:
the backing sheet and the carbon sheet are bound together along a common edge, respectively, to define a binding.

5. The transparent tattoo stencil transfer of claim 4, wherein:
the plastic sheet is positioned between the backing sheet and the carbon sheet prior to the heat transfer process such that it is protected during shipping of the transparent tattoo stencil transfer; and
the plastic sheet is repositioned on top of the carbon sheet during or prior to the heat transfer process.

6. The transparent tattoo stencil transfer of claim 1, wherein:
the plastic sheet has a transmittance value above 50%.

7. The transparent tattoo stencil transfer of claim 1, wherein:
a shipping configuration is defined by the plastic sheet being removably positioned between the backing sheet and the carbon sheet; and
a heat transfer configuration is defined by the plastic sheet being positioned on top of the carbon sheet.

8. The transparent tattoo stencil transfer of claim 7, wherein:
at least one semi-transparent tissue sheet is positioned above the carbon sheet in the shipping configuration for protecting the carbon sheet.

9. The transparent tattoo stencil transfer of claim 7, wherein:
at least one semi-transparent tissue sheet is removed from between the carbon sheet and the plastic sheet in the heat transfer configuration.

10. The transparent tattoo stencil transfer of claim 1, wherein:
at least one of a white sheet or a semi-transparent tissue sheet is positioned above the carbon sheet.

11. A method of utilizing a transparent tattoo stencil transfer, the method comprising the steps of:
(a) providing the transparent tattoo stencil transfer comprising at least a backing sheet, a carbon sheet positioned above the backing sheet, and a plastic sheet positionable above the carbon sheet;
(b) positioning a tattoo design between the backing sheet and the carbon sheet;
(c) performing a heat transfer process on the transparent tattoo stencil transfer to transfer a carbon image of the tattoo design from the carbon sheet onto the plastic sheet; and
(d) positioning the plastic sheet with the carbon image of the tattoo design onto a client's skin for transferring the carbon image of the tattoo design for tattooing.

12. The method of claim 11, wherein step (a) further comprises:
repositioning the plastic sheet from a shipping configuration between the backing sheet and the carbon sheet to a heat transfer configuration on top of the carbon sheet.

13. The method of claim 11, wherein:
the transparent tattoo stencil transfer further comprises at least one semi-transparent tissue sheet positioned above the carbon sheet for protecting the carbon sheet in a shipping configuration.

14. The method of claim 13, wherein, prior to step (c), the method further comprises:
removing the at least one semi-transparent tissue sheet.

15. The method of claim 13, wherein:
two or more of the backing sheet, the carbon sheet, or the at least one semi-transparent tissue sheet are bound together along a common edge, respectively, to define a binding.

16. The method of claim 13, wherein:
the binding comprises glue.

17. The method of claim 11, wherein step (c) further comprises:
feeding the transparent tattoo stencil transfer with the tattoo design through a thermofax machine.

18. The method of claim 17, further comprising:
positioning the transparent tattoo stencil transfer with the tattoo design into a thermo carrier prior to feeding the transparent tattoo stencil transfer with the tattoo design through the thermofax machine.

19. The method of claim 11, wherein step (d) further comprises:
removing the plastic sheet with the carbon image from the transparent tattoo stencil transfer.

20. The method of claim 11, wherein step (d) further comprises:
transferring the carbon image from the plastic sheet into the client's skin.
